(19) United States
(12) Patent Application Publication
(10) Pub. No.: US 2015/0051450 A1
(43) Pub. Date: Feb. 19, 2015

(54) SYSTEM, DEVICE AND METHOD FOR MEASUREMENT OF ESOPHAGEAL WALL BLOOD PERFUSION

(71) Applicant: The Regents of the University of California, Oakland, CA (US)

(72) Inventors: Ravinder Mittal, La Jolla, CA (US); Valmik Bhargava, San Diego, CA (US)

(21) Appl. No.: 14/385,757

(22) PCT Filed: Apr. 1, 2013
(86) PCT No.: PCT/US2013/034864

§ 371 Date: Sep. 16, 2014

Related U.S. Application Data
(60) Provisional application No. 61/617,712, filed on Mar. 30, 2012.

(51) Int. Cl. A61B 5/00; A61B 17/34; A61B 5/145; A61B 8/12; A61B 5/026; A61B 5/0205

(52) U.S. Cl. CPC A61B 5/6852; A61B 5/0261; A61B 5/0205; A61B 5/14539; A61B 5/14542; A61B 8/12; A61B 17/3468; A61B 5/7475; A61B 5/0538
USPC 600/301; 600/478

(57) ABSTRACT

The present invention provides a device and system useful as a new diagnostic modality to differentiate the etiology of heartburn/chest pain by measuring esophageal blood flow. Furthermore, the device and system of the present invention can be used as a new diagnostic modality to differentiate the etiology of abdominal pain by measuring blood in the wall of gastrointestinal tract including rectum and colon blood flow.

### FEDERAL FUNDING LEGEND

This invention was made with governmental support under a Grant Number RO-1 awarded by the National Institutes of Health. The government has certain rights in the invention.

### BACKGROUND OF THE INVENTION

1. Field of the Invention

The present invention relates generally to the fields of gastroenterology and esophageal biology. More specifically, the invention relates to the monitoring of the esophageal gastrointestinal tract wall blood perfusion and uses thereof.

2. Background Information

Non-cardiac “angina like pain” of esophageal origin and heartburn, nonresponsive to potent acid inhibition therapy are major health problems for which there are no satisfactory therapies. Angina like esophageal pain accounts for 26% of all emergency room visits in the United States and costs billions of dollars to the healthcare system. With the availability of potent acid inhibitors (proton pump inhibitors), it is clear that in 50-70% of patients with gastroesophageal reflux disease (GERD) like symptoms, acid is not the cause of heartburn. Based on the current paradigm, esophageal pain and proton pump inhibitor unresponsive heartburn are caused by esophageal hypersensitivity (allodynia & hyperalgesia). Patients with “angina like pain” demonstrate hypersensitivity to distension by balloon.

Hypersensitivity can also be to normal physiological stimuli such as normal contractions, normal amount of acid reflux or other stimuli that do not cause pain under normal conditions. In the experimental setting, electrical stimulation in the distal esophagus causes sensitization of proximal esophagus. Repeated acid infusion into the esophagus and repeated distension of the esophagus with balloon also lead to exaggerated sensory responses in normal subjects and esophageal pain patients.

One of the suspected causes of chest pain is esophageal muscle spasm. However, some studies that utilized prolonged ambulatory manometry and pH recordings, conducted in the 1980's and early 1990s, failed to identify abnormal motor events at the time of pain. Using simultaneous ultrasound imaging and manometry, a unique contraction “sustained esophageal contraction (SEC)” was found in association with esophageal pain and heartburn. The sustained esophageal contraction was later determined to be prolonged during contraction of longitudinal muscles of esophagus without concurrent circular muscle contraction. Intraluminal manometry cannot monitor longitudinal muscle contraction or sustained esophageal contraction. One can make several observations with regards to the patterns of longitudinal muscle contraction in health and disease using Ultrasound (US) imaging. However, recording and analysis of US images, especially over extended time is expensive, tedious and labor intensive. As a result, other laboratories have not studied longitudinal muscle and its relationship to esophageal pain. High resolution manometry (HRM) is a technique to record circular muscle contraction during peristalsis and can also be used to record longitudinal muscle contraction related shortening of the esophagus.

Blood flow in the myocardium occurs predominantly during the diastolic phase of the cardiac cycle, because myocardial contraction during systole restricts entry of blood in the muscle. Along the same lines, contraction in a limb muscle restricts blood flow into the muscles. In a visceral smooth muscle, i.e., gastric muscle, the blood perfusion diminishes during gastric contraction, as visualized by constriction of blood vessels using in-vivo microscopy. In another study in which laser Doppler flowmetry was used to study the esophageal wall perfusion, esophageal muscle contraction induced by vagus nerve or local muscle stimulation was seen to reduce esophageal wall perfusion in rats.

Several investigators have studied esophageal wall perfusion in relationship to the esophageal pain using a esophageal rewarming rate by thermister technique. Another study, studied the relationship between esophageal distension by a balloon and alteration in the wall blood perfusion using laser a Doppler probe in a rat. In order to monitor esophageal wall blood perfusion by the laser Doppler technique, the probe has to be firmly anchored to the esophageal wall because relative movement between the esophageal wall and the laser Doppler probe causes artifact in the recordings. In animal studies, one can anchor the laser Doppler probe to esophageal well using surgically placed sutures, which is obviously not practical for human studies.

Swallow-induced peristaltic contraction of the esophagus is associated with simultaneous contraction of the circular and longitudinal muscles of the esophagus. On the other hand, transient lower esophageal sphincter (LES) relaxation is associated with a selective contraction of the longitudinal muscles of the distal esophagus.

### SUMMARY

The present invention discloses the development of a custom designed laser Doppler probe. This probe, when anchored to the esophageal wall, 3-6 cm above the lower esophageal sphincter in such a manner that the light beam stays directed towards the esophageal wall, there is no motion between the transducer and esophageal wall during peristalsis. The Doppler transducer stays anchored to give continuous monitoring of the esophageal wall blood perfusion. A high-resolution manometry catheter equipped with impedance electrodes can record esophageal pressures and gestroesophageal reflux events. Esophageal contractions induced by dry swallows can result in a 66±4% reduction in the esophageal wall blood perfusion. Similar reduction and relationship with contraction amplitude can be realized with wet swallows. Atropine (IV bolus 6 microgm/kg) can reduce swallow-induced esophageal contraction amplitude and decrease esophageal wall blood perfusion reduction in association with contractions. Transient lower esophageal sphincter relaxations can also be associated with reduction in the esophageal wall blood perfusion, albeit of smaller amplitude but longer duration as compared to swallows. Thus, the present disclosure provides an innovative technique for recording esophageal wall blood perfusion for extended time periods in humans; 2) contraction of circular and longitudinal muscle during peristalsis and selective longitudinal muscle contraction during transient lower esophageal sphincter relaxation can cause reduction in the esophageal wall blood perfusion; and 3) using this innovative technique, one may determine that esophageal wall ischemia is the cause of esophageal pain.

The above methodology can be applied to study blood flow in all parts of the gastrointestinal tract, the probe can be anchored to stomach, small intestine, large intestine and rectum to study blood flow in these areas. Ischemia of the wall of the gastrointestinal tract can be the cause of abdominal pain in different regions of the abdomen.

Thus, the present disclosure is directed to blood flow measurements in the wall of the entire gastrointestinal tract, either in the stationary setting (patient being monitored in laboratory setting) or in the ambulatory setting, an ambulatory portable device with a GI wall anchoring system to measure blood flow and physiological signals in the GI track tissue. Various probes and sensors can be incorporated into the anchoring system to measure multiple signals simultaneously in the tissue to which it is attached. For example, a laser Doppler for blood flow probe, ultrasound probe for imaging longitudinal muscle contraction (function), and various sensors for pH, impedance, oxygen and/or carbon dioxide can be incorporated into the embodiments of the system described in the present disclosure. The probe/sensor bundle contains a suction cup which will suck a small portion of the mucosal tissue, once it is placed in the appropriate position. The device is then attached to the tissue by pinning it. Upon completion of testing, it can be removed by pulling back on the pinning element. The device can be left attached to the GI wall for prolonged periods of time (more than a few hours) while the subject conducts normal activity. When an event, such as heartburn or chest pain or other symptoms occurs that causes reduction in blood flow, the subject can identify it on the ambulatory portable recorder. Upon completion of testing, the subject returns to the laboratory and the data from the recorder can be downloaded and reviewed to confirm the cause of symptoms. The present disclosure, therefore, provides both a portable acquisition device and an anchoring system for the probe(s)/sensor(s) so that it can stay attached to the GI wall for a prolonged time period to allow continuous monitoring of the blood flow and other signals.

It is anticipated that the present disclosure will have various applications. For example, the device permits an investigator to study whether local ischemia is associated with the symptoms. Thus, the device and system of the present disclosure can be used as a new diagnostic modality to differentiate the etiology of heartburn/chest pain or other systems by measuring esophageal blood flow. Furthermore, the device and system of the present disclosure can be used as a new diagnostic modality to differentiate the etiology of abdominal pain by measuring rectum and colon blood flow. The device and system of the present disclosure can be used as a tool to find new treatments and test the efficacy of these treatments for the above clinical conditions.

The device and system of the present disclosure describes a prototype with a laser Doppler probe and anchoring system which can be used for the successful measurement of esophageal wall perfusion during esophageal contraction and transient lower esophageal sphincter relaxations in humans. The results can demonstrate that esophageal contraction reduces esophageal wall perfusion.

Other and further aspects, features, benefits, and advantages of the present invention will be apparent from the following description of the various embodiments of the disclosure.

### BRIEF DESCRIPTION OF THE DRAWINGS

So that the matter in which the above-recited features, advantages and objects of the disclosure, as well as others which will become clear, are attained and can be understood in detail, more particular descriptions and certain embodiments of the disclosure briefly summarized above are illustrated in the appended drawings. These drawings form a part of the specification. It is to be noted, however, that the appended drawings illustrate various exemplary embodiments of the disclosure and therefore are not to be considered limiting in their scope.

FIGS. 1A-1E illustrate schematic figures of an exemplary laser Doppler probe and use of the exemplary laser Doppler probe. FIG. 1A is a schematic illustration of an exemplary laser Doppler probe. FIG. 1B is a photographic representation of the exemplary Doppler probe with scale in background; FIGS. 1C and 1D are schematic illustrations of a Bravo and laser Doppler probe anchored to the esophageal wall with the Laser Doppler probe taped to a Bravo pH capsule. Using the Bravo pH delivery system (FIG. 1C) the capsule can be anchored to the esophageal wall and the delivery system can be removed (FIG. 1D). FIG. 1E illustrates exemplary Laser Doppler perfusion recordings when the capsule was inadequately fixed to the esophageal wall (poor contact) and when properly anchored.

FIGS. 2A-2C illustrate Esophageal wall perfusion during swallow induced esophageal contractions. Doppler perfusion tracings are superimposed (white lines) on high resolution manometry (HRM) plots. The Doppler probe can be taped to the Bravo pH capsule which can be anchored 4 to 6 cm above the lower esophageal sphincter. In FIG. 2A, note the fall in laser Doppler perfusion with each swallow which accompanied esophageal contraction. Amplitude and duration of the perfusion record can be related to the amplitude and duration of esophageal contraction. In FIG. 2B, note the effect of wet swallows which induced esophageal wall contraction on the reduction in esophageal wall perfusion. FIG. 2C shows the effect of atropine on the laser Doppler perfusion during wet swallow. Note, atropine reduced the esophageal contraction amplitude and the associated reduction in blood perfusion with esophageal contractions.

FIGS. 3A-3B illustrate the effect of esophageal contraction duration and amplitude on the esophageal wall perfusion. FIG. 3A shows an exemplary plot of contraction duration measured from HRM versus duration of reduction in perfusion measured from Doppler signal. FIG. 3B shows an exemplary plot of contraction amplitude versus percent reduction in blood perfusion.

FIGS. 4A-4C illustrate esophageal wall perfusion during Transient lower esophageal sphincter relaxation. Three examples of esophageal wall perfusion during transient lower esophageal sphincter relaxations are shown. Note, reduction in esophageal wall perfusion with transient lower esophageal sphincter relaxation in FIG. 4A and FIG. 4B but not in FIG. 4C. Also note that wall perfusion reduction can be less during transient lower esophageal sphincter relaxation as compared to esophageal contractions. The tracings represent impedance recording and show gastroesophageal reflux during transient lower esophageal sphincter relaxation.

FIGS. 5A-5B: show a comparison of reduction in perfusion during esophageal contraction and transient lower esophageal sphincter relaxation. FIG. 5A shows average percent reduction in peak esophageal wall perfusion associated with wet swallows (WS), dry swallows (DS) and transient lower esophageal sphincter relaxations (TLESR). FIG. 5B shows the average duration of esophageal wall blood perfusion reduction associated with wet swallows, dry swallows and transient lower esophageal sphincter relaxations. Wet and dry swallow can result in less reduction in perfusion compared to transient lower esophageal sphincter relaxations and the reduction in perfusion duration can be significantly longer compared to TLESR.

FIGS. 6A-6B illustrate heartburn and esophageal wall perfusion. FIG. 6A shows esophageal wall blood perfusion during wet swallows and 3 different heartburn episodes (FIG. 6B). Note, the duration of reduction in perfusion can be longer with heartburn episodes than with the wet swallows.

FIG. 7 illustrates a front view of a probe and delivery system according to one embodiment of the subject disclosure.

FIG. 8 illustrates a side view of the probe of FIG. 7 in accordance with one embodiment of the subject disclosure.

FIGS. 9A-C illustrate side views of the probe of FIG. 7 as it is being installed in a patient according to one embodiment of the subject disclosure.

FIG. 10 illustrates one embodiment of an ambulatory system employing a probe according one embodiment of the subject disclosure.

### DESCRIPTION OF EXEMPLARY EMBODIMENTS

It is one aim of the present invention to determine the temporal correlation between esophageal pain/heartburn and esophageal longitudinal muscle contraction with blood flow to the esophageal well using a novel system that anchors a laser Doppler probe to the wall of the esophagus. Using the described system it will be possible to determine the temporal correlation between spontaneous symptoms in gastrointestinal tract and blood flow to the wall of gastrointestinal tract.

The relationship between muscle contraction and blood flow in the esophageal wall or anywhere in GI tract is relatively unstudied. The present disclosure shows that an esophageal contraction causes reduction in the esophageal wall blood flow (perfusion). Prolonged esophageal muscle contraction is likely to cause prolonged reduction in the esophageal wall perfusion thus raising the possibility of esophageal wall ischemia as a cause of esophageal pain.

It is another of the present disclosure to use laser Doppler blood flow measurements to measure the effect of muscle contraction on blood flow (perfusion) in the esophagus and entire GI tract in humans. Since esophageal contraction associated with chest pain is a prolonged longitudinal muscle contraction, it is possible that the latter reduces wall perfusion and produces ischemia to cause pain.

Swallow-induced peristaltic contraction is associated with simultaneous contraction of circular and longitudinal muscles of the esophagus. On the other hand, transient lower esophageal sphincter relaxation is associated with a selective contraction of the longitudinal muscles of the distal esophagus. Using a novel procedure according to the present disclosure, a laser Doppler probe can be anchored to the esophageal wall to determine blood flow (wall perfusion) in the esophageal wall during peristaltic contractions and TLESR in humans.

It is one aim of the present invention to determine the temporal correlation between longitudinal muscle spasm, esophageal wall ischemia and esophageal pain and to determine if chest pain and heartburn symptoms are associated with esophageal wall ischemia secondary to the sustained contraction of the longitudinal muscle.

In summary, esophageal longitudinal muscle spasm induces ischemia of esophageal wall, which in turn causes esophageal pain. Novel techniques of ambulatory HRM, laser Doppler flow measurements and creative experimental designs are described herein.

The following examples are given for the purpose of illustrating various embodiments of the disclosure and are not meant to limit the present invention in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Experimental Design

Studies were conducted in the 12 healthy volunteers (mean age 32.5 years, range 18-51, 11 males). Protocol for the studies was approved by the “University of California San Diego Institutional Review Board for the Protection of Humans” and each individual gave a written consent prior to enrollment in the study. Subjects fasted for at least 6 hours prior to the commencement of the study.

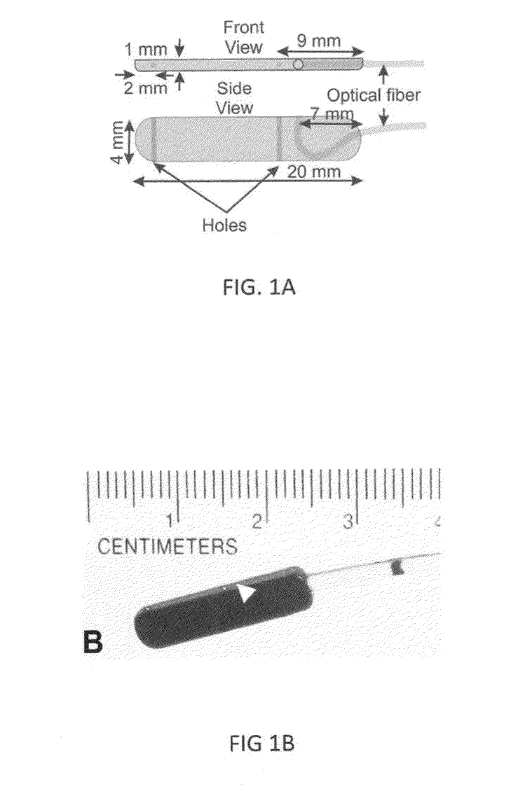

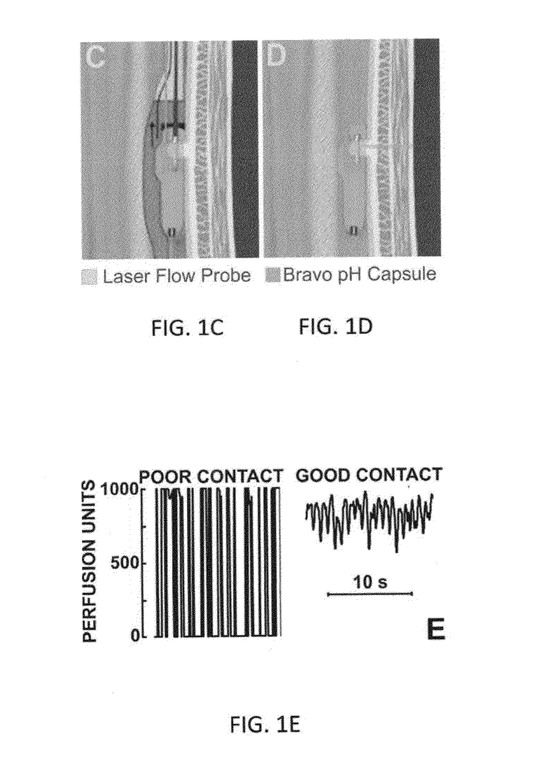

Esophageal wall perfusion can be monitored using a custom designed laser Doppler probe (FIGS. 1A and 18) that can be taped to a Bravo pH monitoring system and the two together can be anchored to the esophageal wall. A wireless Bravo pH monitoring technique makes it possible to anchor the pH capsule to the esophageal mucosa. Through a vacuum, connected to the cup of a Bravo pH capsule by the delivery system, a small volume of mucosa can be sucked into the cup of Bravo pH capsule. Using a mechanical system located in the handle of delivery system, a pin can be inserted through the esophageal mucosa in the suction cup that anchors the pH capsule to the esophageal wall (FIGS. 1C and 1D) for approximately 5 days or more.

### EXAMPLE 2

### Laser Doppler Probe & Catheter

A unique aspect of the laser Doppler probe of the present disclosure is its relatively thinness, i.e., 1 mm wide. The probe can be 4 mm deep and 20 mm in length (FIGS. 1A and 1B) and can be connected to the laser Doppler perfusion monitor via a 1.5 mm diameter, 150 cm long fiber optic cable. The laser Doppler probe can be firmly taped to the Bravo pH capsule using paraffin film (FIGS. 1C and 1D) in a fashion so that when Bravo pH capsule is anchored to the esophageal wall so is the laser Doppler probe. The laser beam exits from the laser Doppler probe in the direction of esophageal wall, and at the level of the suction cup in the Bravo pH capsule. The combined dimensions of the Bravo capsule and laser probe can be as small as 5×4 mm or less, approximately the size of manometry catheters use in clinical practice (4-5 mm in diameter).

The laser Doppler probe taped to the Bravo capsule and delivery system can be passed through the nose in patients. In patients in which this method does not work because of the inflexibility of Bravo pH Capsule making it difficult to negotiate the catheter assembly through nose, an alternative strategy can be used. The laser Doppler probe (1.5 mm diameter) can be first passed through the nose and probe can be pulled out from the mouth. Laser Doppler probe can then be taped to the Bravo pH capsule as described earlier. The combined laser Doppler probe and Bravo capsule assembly can then be introduced through the mouth into the esophagus. The Bravo capsule can be deployed (using a routine method) at 4-6 cm above the lower esophageal sphincter (LES) and the delivery system can then be removed thus leaving behind the laser Doppler catheter exiting from the nose, as shown in FIG. 1. Using this approach, the laser light beam stays directed towards the esophageal mucosa for the entire duration of the study. The laser beam is unaffected by the luminal contents and relative movement between the laser beam and esophageal wall is prevented during esophageal contractions and respiration.

Following placement of the Bravo pH capsule and the laser Doppler probe catheter, a high resolution manometry (HRM) catheter equipped with multiple impedance electrodes can be placed in the esophagus, through either the same nostril as the laser Doppler probe or the contralateral nostril. Thus, simultaneous and continuous recordings of the pressures (esophagus, lower esophageal sphincter and stomach), intraluminal impedance at multiple levels in the esophagus, esophageal pH and esophageal wall blood perfusion can be obtained.

Following baseline recordings for 10-15 min, 5 dry and 8-10 wet swallows (5 ml water) can be recorded. Subjects then can be directed to eat a standardized 400 Kcal soft meal. Recording can be performed for an additional one hour, part of the time in the upright and the remainder in supine position. Finally, atropine 7.5 μg/kg can be injected intravenously, following which 8-10 wet swallow-induced esophageal contractions can be recorded. This exemplary study protocol can take approximately 3 hours to complete. At the end, the laser Doppler probe can be pulled out by applying a gentle tug to the catheter, which detaches the laser Doppler probe from the Bravo pH capsule. In cases where the Doppler probe and Bravo pH capsule detach from the esophageal wall as one piece and cannot be pulled through the nose, the Bravo capsule and laser Doppler probe can be pulled through the mouth and the pH capsule can be detached from the laser Doppler probe when pulled out.

### EXAMPLE 3

Data Analysis & Statistical Methods

In addition to the laser Doppler perfusion units (PU) signal, the Doppler monitor can also provide continuous record of the Total Backscatter (TB) of the laser light signal. Total Backscatter can be an indicator of the relative movements between the esophageal mucosa/wall and laser Doppler probe. Total backscatter can be essentially a reading of how much light is reflected back to the probe/instrument. Total backscatter signal reading of greater than 1 and a relatively flat line can indicate that the measurement conditions were stable and there is minimal to no relative movement between the tissue and the probe. An esophageal blood perfusion signal can be temporally filtered using a 2 seconds moving time average filter. Duration of esophageal blood flow reduction can be determined by the interval over which the perfusion drops by more than 10% of the baseline values. Baseline pressures and esophageal blood flow can be averaged over a 10 s interval prior to an event. Baseline data prior to eating and 5-10 minutes after the meal can be averaged over a 30 to 150 s interval, avoiding dry swallow-induced esophageal contraction. Contraction pressure amplitude can be determined based on the maximum pressure in the region of the Doppler probe during wet and dry swallows. The laser Doppler perfusion value just before and at the peak of esophageal contraction can be determined and the percent reduction in the perfusion value can be calculated. Transient lower esophageal sphincter relaxation periods can be identified based on a predefined criteria, i.e., long duration, complete lower esophageal sphincter relaxations, not initiated by swallow and perfusion values (percent reduction) can be calculated.

Mean values and standard error of mean (SEM) can be reported. Comparisons can be done using one-way ANOVA (and nonparametric) and unpaired t-test. Statistical significance can be defined as p<0.05.

### EXAMPLE 4

### Effect of Laser Doppler Contact with Esophageal Wall Blood Perfusion Recording

The total backscatter value can be continuously recorded by the laser Doppler system and can provide a gauge of the adequacy of contact between the laser light beam and the esophageal mucosa. Values greater than 1 and a flat line suggest good contact. FIG. 1E shows examples of poor contact (left side) with a total backscatter value of less than 1. This recording is generally unstable with wide fluctuations of esophageal wall blood perfusion values. On the other hand, when contact between the laser Doppler beam and esophageal mucosa is good, as suggested by the total backscatter recording values of more than 1 and a flat line recording (right side FIG. 1E), esophageal wall blood perfusion recording can be stable with rhythmical fluctuations of small amplitude. These fluctuations can be temporally related either with the heart beat or the respiration.

### EXAMPLE 5

### Baseline Esophageal Wall Perfusion Values

Baseline esophageal perfusion values, i.e., in between esophageal contractions, can be measured to range from 413 to 938 perfusion units (PU) and can be normally distributed. Mean baseline esophageal blood perfusion values among various patients can be measured as 701±63 PU. Ingestion of the meal generally does not cause significant change in the baseline esophageal wall perfusion values.

### EXAMPLE 6

### Esophageal Wall Perfusion During Swallow-Induced Esophageal Contraction

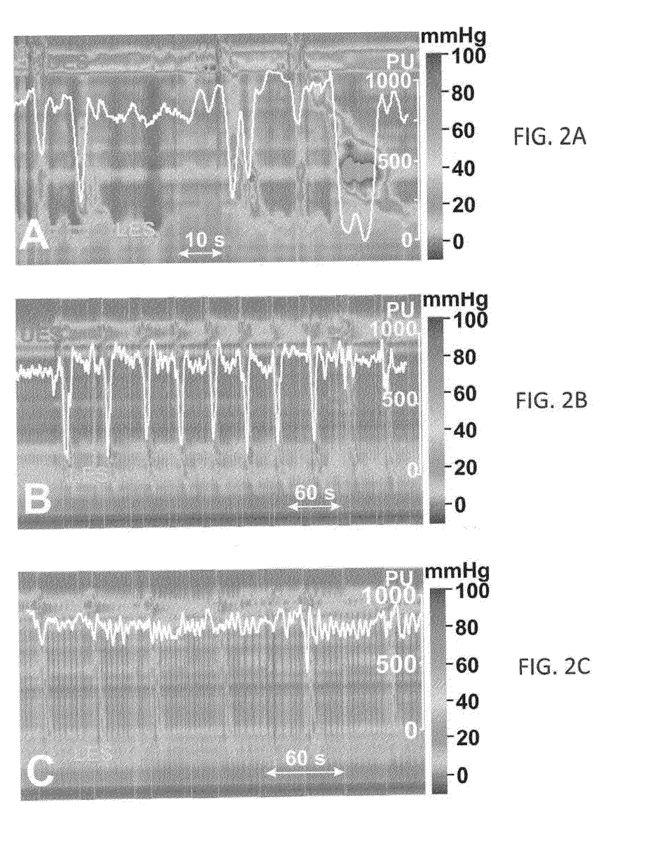

FIG. 2A shows the effects of dry swallow induced esophageal contractions on the esophageal wall perfusion. Note that each contraction can result in a drop in the esophageal wall blood perfusion values. Also note that the reduction in the esophageal wall blood perfusion can be greater when the contraction amplitude is higher. Furthermore, the duration of esophageal wall perfusion reduction can be similar to the duration of esophageal contraction.

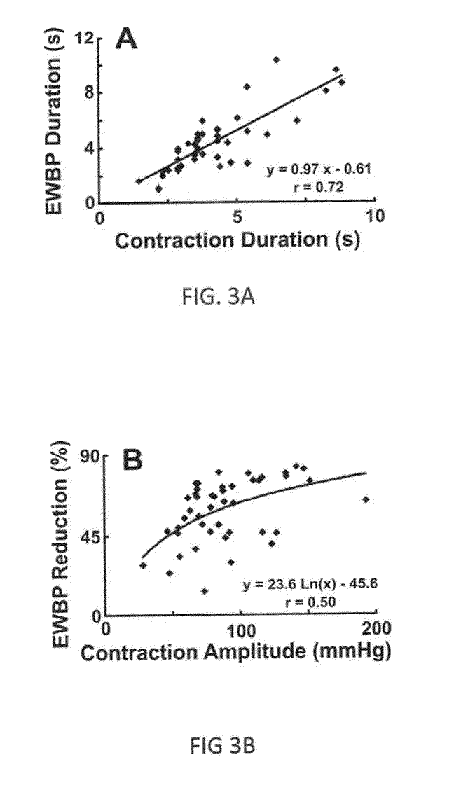

FIG. 2B shows exemplary effects of wet swallow-induced contractions on the esophageal wall perfusion. The reproducibility of the decrease in perfusion signal with swallows can be seen. In general, wet swallows can induce contraction resulting in an approximately 60% reduction in the esophageal wall perfusion. The effect of dry swallow induced contraction on the esophageal perfusion can be somewhat less but not significantly different from the wet swallow induced contraction. Atropine can cause significant reduction in the swallow-induced esophageal contraction amplitude and it also can decrease the reduction in esophageal wall perfusion associated with esophageal contractions, (FIG. 2C). There can be a significant relationship between the duration of contraction and duration of reduction in the esophageal wall perfusion (FIG. 3A) (r value of 0.72 and regression equation: y=0.9674 x−0.6071, where y is the duration estimated from the perfusion values and x is the duration from high resolution manometry in patients with a total of 48 swallows). There can also be a significant relationship between contraction pressure and percent reduction in esophageal wall blood perfusion with r value of 0.50 for patients (39 swallows) showing in FIG. 3B. The best fit for this relationship can be a curviliner line and not a straight line.

### EXAMPLE 7

### Esophageal Wall Perfusion During Transient LES Relaxation

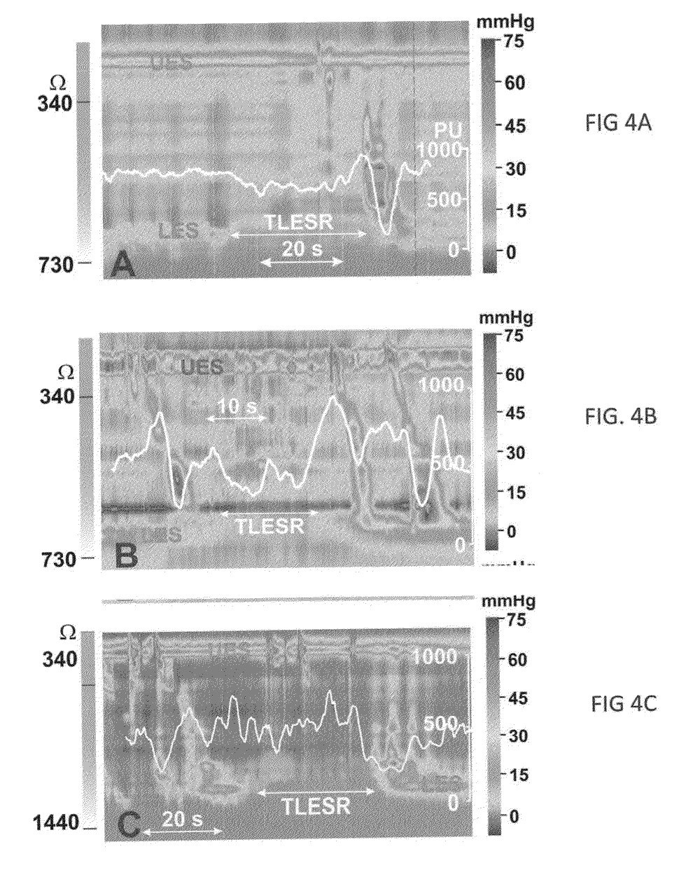

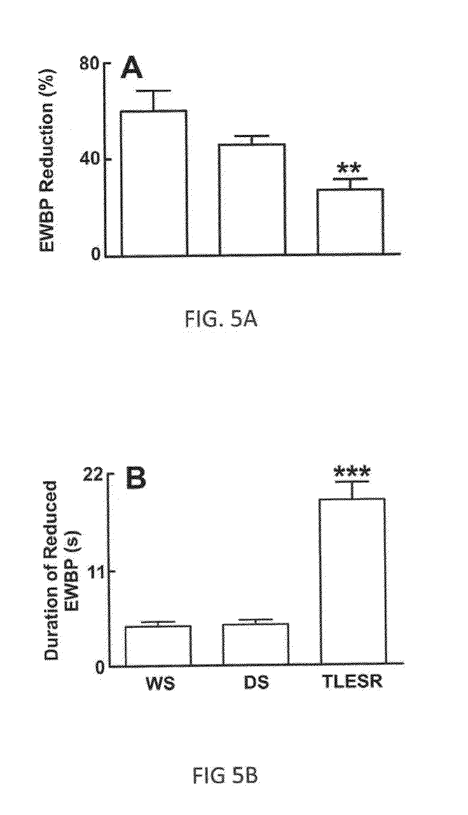

Decrease in esophageal wall perfusion can also be observed during periods of transient lower esophageal sphincter relaxations. This decrease can start at the onset of transient lower esophageal sphincter relaxation and persist throughout the period of relaxation (FIGS. 4A, 4B and 4C). Peak reduction can be seen with the esophageal contraction at the termination of transient lower esophageal sphincter relaxation. The decrease in perfusion during transient lower esophageal sphincter relaxation can be variable, and can be seen to range from 0-64%, with a mean reduction of 29±3% (FIGS. 5A and 5B). The duration of reduction can be 18.9±2 s and can be significantly longer than the duration of reduction associated with wet swallows induced contraction (4.6±0.5 s,) and dry swallow induced esophageal contraction (4.7±0.6 s) (FIGS. 5A and 5B). Some of the patients can demonstrate acid reflux episodes (pH<4) during the recording period. There generally is no measurable difference in the esophageal wall blood perfusion values between those transient lower esophageal sphincter relaxations that were associated with acid reflux compared to the ones that were not associated with acid reflux (21±6 vs 24±5%).

### EXAMPLE 8

### Esophageal Wall Perfusion and Heartburn

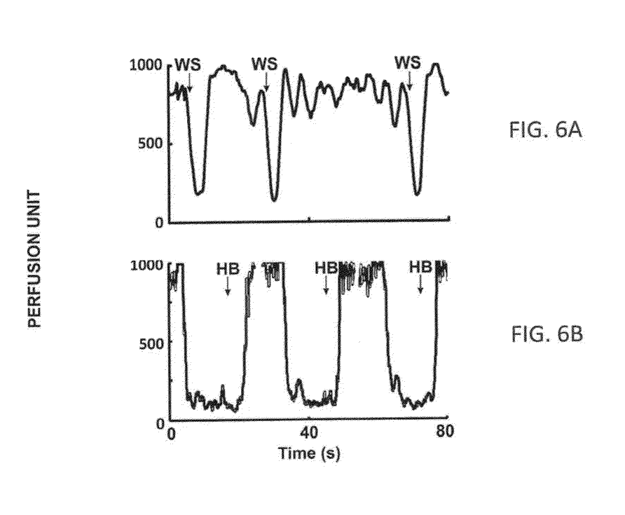

In one study, one subject complained of 3 transient heartburn symptoms during the recording period even-though he never experienced such symptoms routinely. Each of these heartburn symptoms was preceded by a 20 second period of complete stoppage of esophageal wall perfusion, followed by spontaneous recovery to the baseline (FIGS. 6A-6B). The esophageal wall blood perfusion during heartburn complaint was significantly lower than with WS or DS, 91±9.7 vs. 87±10.8%, p=0.034. The HRM catheter was not placed into the esophagus in this subject and thus no manometric events associated with the heartburn are available.

### DISCUSSION

In summary, the present disclosure shows that it is possible to monitor esophageal wall blood perfusion continuously and for extended time periods using the laser Doppler flowmetry technique. Thus, the present invention describes a novel laser Doppler probe anchoring technique to the esophageal mucosa. Esophageal contractions can be associated with a reduction in the esophageal wall perfusion, the degree and duration of reduction of which can be directly related to the amplitude and duration of esophageal contraction. Atropine can reduce the esophageal contraction amplitude and can decrease the reduction in esophageal wall perfusion associated with esophageal contraction. Transient lower esophageal sphincter relaxation can be associated with a reduction in the esophageal wall perfusion, albeit of smaller amplitude but longer duration than the swallow-induced esophageal contraction.

Esophageal blood flow has been studied using various techniques in both animal and human studies. Using microsphere injection techniques, it can be determined that the blood flow to the opossum lower esophageal sphincter is greater than to the body of the esophagus. Furthermore, the mucosa and submucosa receive greater blood flow compared to the muscularis propria and there is a gradient of blood flow in the esophagus; the distal esophagus receives greater blood flow than the proximal. Animal studies also show that acid instillation into the esophagus increases esophageal mucosal blood flow through the release of histamine, nitric oxide, and calcitonin gene-related peptide. The thermistor technique that records rewarming of the cold water injected into the esophageal lumen (as a surrogate of esophageal blood flow) can be used in the humans. It has been found that a significantly longer warming rate of injected water in patients with esophageal spasm compared with 20 controls and proposed esophageal wall ischemia as the cause of pain. A computerized thermistor recording has been used and found an increase in the mucosal blood flow during acid infusion into the esophagus in patients responding to acid-infusion with heartburn. The studied patients who responded to edrophonium injection with/without pain (a test of esophageal pain) were also studied. No difference in the rewarming rates was detected between the pain-positive and pain-negative patient groups. It's likely that the thermistor/rewarming technique is not adequate to study the esophageal blood flow because retention of even a small amount of water in the esophagus due to inadequate clearance may affect esophageal rewarming significantly. Laser Doppler perfusion monitoring has been used to determine the distension-related esophageal pain and it was found that the esophageal wall stress and strain, rather than wall ischemia is the cause of distension related pain. In this study, the laser Doppler probe was placed inside a balloon. No mention of the adequacy of contact between the laser Doppler beam and the esophageal mucosa was made in the above studies. Total backscatter values, recorded continuously by the laser Doppler system can provide an important parameter of the adequacy of contact between the laser light beam and the esophageal mucosa. The stable esophageal wall perfusion values are generally only obtained when the total backscatter values were greater than one.

No studies of the relationship between esophageal muscle contraction and esophageal wall perfusion in humans are known. These findings in the current study are similar to a recently reported rat study, i.e., esophageal muscle contraction reduces esophageal wall perfusion. Amplitude and duration of reduction in the esophageal wall perfusion can be directly related with the amplitude and duration of esophageal contractions. Atropine can reduce esophageal contraction amplitude and associated an decrease in the esophageal wall perfusion can be observed. Transient lower esophageal sphincter relaxation can be associated with a distinct pattern of longitudinal muscle contraction that starts in the distal esophagus and traverses in an anti-peristaltic fashion toward the oral end. The duration of longitudinal muscle contraction associated with transient lower esophageal sphincter can be similar to the duration of transient lower esophageal sphincter relaxation. The present disclosure shows that transient lower esophageal sphincter relaxation can also associated with a reduction in the esophageal wall perfusion, albeit of smaller amplitude than the swallow-induced esophageal contraction. The duration of reduction in the esophageal wall perfusion can be similar to the duration of the lower esophageal sphincter relaxation, thereby proving that the longitudinal muscle contraction of the esophagus can reduce esophageal wall perfusion.

The laser Doppler perfusion technique can provide average blood perfusion values in the area of tissue illuminated by the laser light beam. The depth of penetration of the laser beam can generally be felt to be 1.5 m, however in certain tissues it can be up to 5 mm (20). Therefore, laser Doppler recordings generally cannot distinguish the precise site of blood perfusion, mucosa vs the muscle layer. It is likely that the decrease in the esophageal wall perfusion with contraction occurs in all the layers of esophagus because reduction is related to mechanical compression of the vessels by muscular contraction. The effects of contraction of the limb skeletal muscle (gastrocnemius) on the arterial blood flow using a hot wire anemometer have been reported. They describe four phases of changes in the arterial blood flow with the onset of muscle stimulation: 1) back thrust of blood in the artery; 2) diminution or arrest of the blood inflow into the muscle; 3) overshoot of blood flow during relaxation; and 4) the hyperemic after-effect. The first two changes in the arterial blood flow are due to mechanical compression of the blood vessels by the contracting muscle fibers. The compression of blood vessels with muscular contraction has also been observed in the heart and various other skeletal muscles (including diaphragm), as well as the smooth muscles of small intestine. In-vivo microscopy has been used and can provide visual evidence of the constriction of capillaries of the gastric corpus and antrum during electrical field stimulation of the gastric corpus and antrum. Changes in capillary size paralleled that of the spontaneous phasic contractions of gastric corpus and gastric antrum. The decrease in the capillary size can be much greater in the antrum than gastric corpus. The 3rd and 4th phase hyperemia following muscle contraction has been explained on the basis of a peripheral vasodilator action of locally produced metabolites.

The clinical relevance of these findings is that reduction in the esophageal wall perfusion or tissue ischemia is a potent stimulus for the activation of visceral nociceptors and pain. Myocardial ischemia causes angina, dyspepsia and heartburn. Intestinal ischemia is an important cause of acute abdominal pain. Studies show that the intestinal ischemia results in generation of lactic acid, bradykinin, histamine, serotonin, prostaglandins, endothelins and possibly other chemicals that are potent stimuli for the nociceptive nerve endings of the sympathetic nervous system that likely mediate pain. The activation of both spinal and vagal visceral nerve endings in response to intestinal ischemia has been reported. Ischemia of the esophageal wall, as a cause of “angina like” pain has been suspected, as described earlier, but never clearly proven. Human studies that utilize thermister/esophageal re-warming rate have provided conflicting results, possibly because of the inadequate esophageal wall blood perfusion monitoring technique. In some studies, a strong temporal correlation can be found between esophageal pain and heartburn with a sustained esophageal muscle contraction. The latter can be determined to be selective contraction of the longitudinal muscle of esophagus. In the present disclosure, transient LES relaxation which can be associated with selective contraction of the longitudinal muscle of the esophagus can be associated with a reduction in the esophageal wall blood flow. The mechanism by which long duration longitudinal muscle contraction of the esophagus induces pain may be related to the long duration reduction in esophageal wall perfusion or in other words ischemia of the esophageal wall. The laser Doppler perfusion recording techniques described herein may be used to determine the temporal relationship between esophageal pain/heartburn and reduction in the esophageal wall perfusion, i.e., ischemia of the esophageal wall.

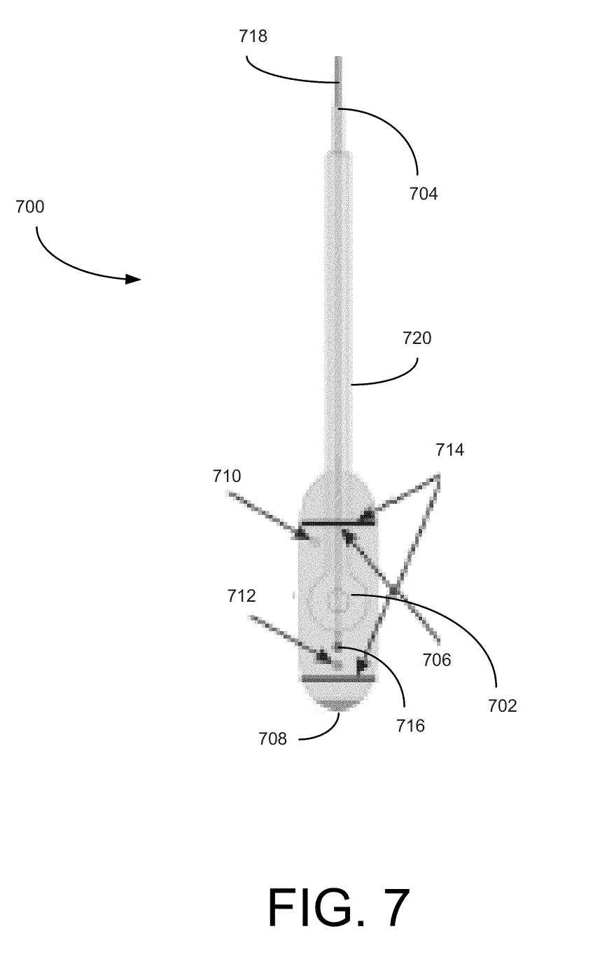

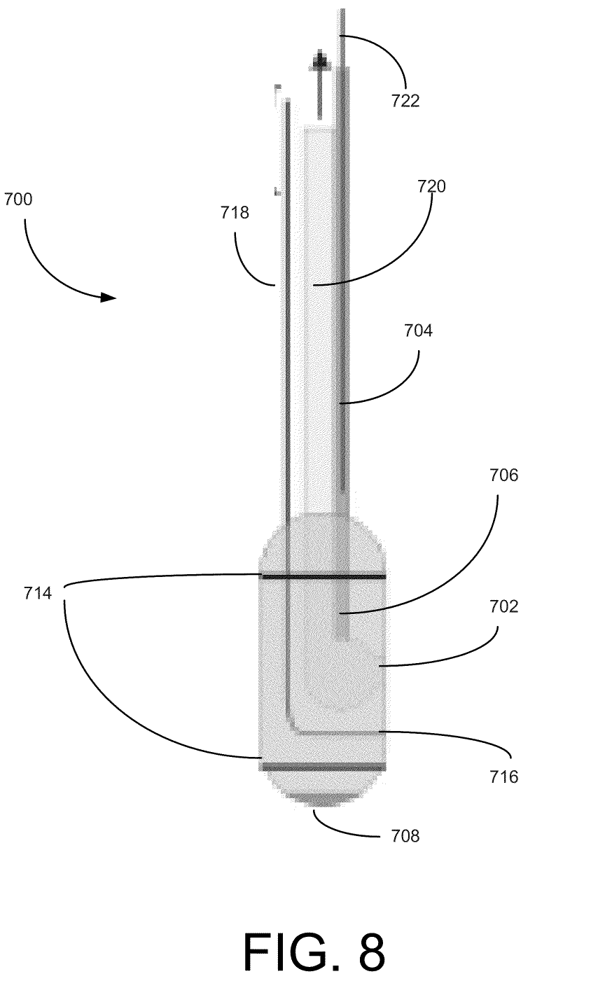

FIG. 7 illustrates one embodiment of a front view of a patient probe 700 for measuring and monitoring esophageal wall blood profusion in a patient. FIG. 8 illustrates a side view of the probe 700 shown in FIG. 7. The patient probe 700 can comprise a suction cup 702 connected to a suction tube 704. The suction tube 704 is configured to create a vacuum at the suction cup 702 causing a small volume of mucosa from the esophageal wall to be sucked into the suction cup 702. A pin 706 can be included, which can be inserted into the mucosa that has been sucked into the suction cup 702 to secure the probe 700 to the esophageal wall. Various sensors can be included on the probe 700 for measuring various aspects of the esophageal wall. For example, the probe 700 can include pH sensor 708, an O₂ or CO₂ sensor 710, an ultrasound (US) transducer 712, impedance rings 714, and/or a laser opening 716. A fiber 718 can be included for funneling laser light to the laser opening 716 and a protective delivery tube 720 can be included for encasing the fiber 718, the suction tube 704, the pin 706, and any electrical wiring 722 needed for the impedance rings 714, US transducer 712, O₂ sensor 710, and/or pH sensor 708. Once the probe 700 is installed in the patient, the delivery tube 720 can be removed.

The probe 700 is configured to allow monitoring of blood flow in the GI tract tissue, e.g., esophageal wall. It is thought that non-cardiac chest pain or heartburn is caused by reduction in blood flow to the esophageal wall caused by prolonged contraction of the muscles in the esophagus. This probe 700 is capable of monitoring relative blood flow in the tissue to which it is attached. The probe 700 is attached to the esophageal wall (by introducing it in to the esophagus through the mouth or nose of the patient) and can be left attached to the wall for prolonged periods of time (more than a few hours) while the patient does his or her normal activity. When an event such as heartburn or chest pain occurs that causes reduction in flow which the patient can identify it on the ambulatory portable recorder. Later on, when the subject returns to the laboratory the data from the recorder can be downloaded and reviewed to confirm the cause of pain/heart burn. Laser Doppler probe itself is not new but we provide an anchoring system to the probe so that it can stay attached to the esophageal wall for prolonged time period to allow continuous monitoring of blood flow.

This device can be built to integrate other physiological signals such as: pH, impedance, oxygen and/or carbon dioxide and ultrasound to document evidence of associated activity such as reflux, longtitudinal muscle contraction.

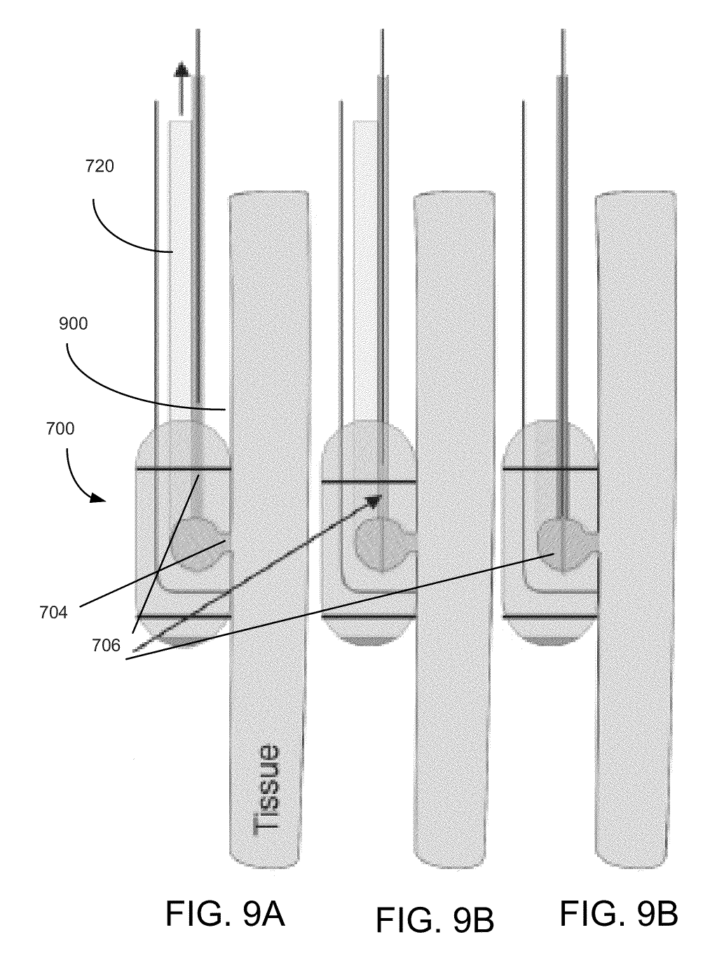

FIGS. 9A-9C illustrate a probe 700 according to one embodiment of the disclosure being installed in a patient. As shown in FIG. 9A, the probe can be fixed to a Bravo pH capsule and passed through the nose of a patient into his/her esophagus. The probe 700 can be guided into place using the protective delivery tube 720. Once in place, for example at 4-6 cm above the lower esophageal sphincter, a vacuum can be created by the suction tube 704 causing the suction cup 702 to suck in mucosa from the esophageal wall temporarily fixing the probe 700 to the esophageal wall 900. The pin 706 can then be inserted into the mucosa inside the suction cup 702 by manipulating the delivery tube 720 as shown in FIG. 9B. Once the pin 706 is in place, the delivery tube 720 can be removed as shown in FIG. 9C.

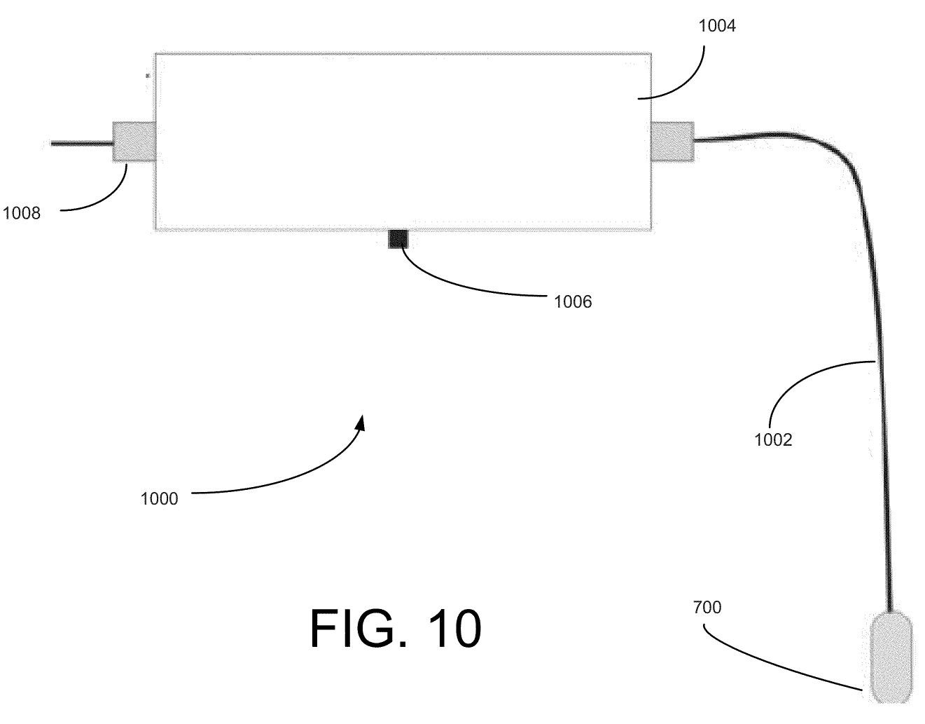

As described above, the probe 700 can be installed in a patient and used as part of an ambulatory recording system, such as the one illustrated in FIG. 10. As shown in the exemplary embodiment illustrated in FIG. 10, a system according to the disclosure can include a probe 700, a fiber optic and electronics interface cable 1002, a portable ambulatory Doppler lase, US, impedance and pH recorder 1004 having an event marker button 1006, and a computer interface 1008. In using this system, the probe 700 is installed in a patient. The fiber optic and electronics interface cable 1002 protrudes from the patient (typically from his/her nose), and is attached to a portable ambulatory Doppler laser, US, impedance and pH recorder 1004 which can be worn by the patient. The recorder 1004 includes a Doppler laser for providing laser light to the probe 700 as well as electronics and recording equipment for recording data from the probe 700. The recorder 1004 can also include an event marker button 1006, which can be used by the patient to record particular events such as a feeling of heartburn, etc. The recorder 1004 records and stores data from the probe 700 also includes a computer interface 1008 for interfacing with a computer (not shown). When connected to a computer via the computer interface 1008, the recorder 1004 can upload the recorded and stored data to the computer via the interface 1008 for further analysis.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

What is claimed is:

1. A device for measuring blood perfusion in the esophageal wall of a patient, the device comprising:
a probe;
a suction cup attached to the probe;
a suction tube connected to the suction cup and configured to produce a vacuum in the suction cup such that a small volume of mucosa from the esophageal wall is sucked into the suction cup;
a pin positioned to be inserted into the small volume of mucosa sucked into the suction cup such that the probe is fixed in place relative to the esophageal wall;
a fiber optic cable configured for delivering a laser beam to and through the probe and in the direction of the esophageal wall such that there is little or no relative movement between the laser beam and the esophageal wall;
wherein the probe is configured to measure laser Doppler perfusion indicative of the blood perfusion of the esophageal wall.

2. The device of claim 1 further comprising a pH sensor.

3. The device of claim 1 further comprising an O₂ sensor.

4. The device of claim 1 further comprising a CO₂ sensor.

5. The device of claim 1 further comprising impedance rings.

6. The device of claim 1 further comprising an ultrasound transducer.

7. The device of claim 1 further comprising a delivery tube for installing the probe in the patient.

8. A system for measuring blood perfusion in the esophageal wall of a patient, the device comprising:
a probe;
a suction cup attached to the probe;
a suction tube connected to the suction cup and configured to produce a vacuum in the suction cup such that a small volume of mucosa from the esophageal wall is sucked into the suction cup;
a pin positioned to be inserted into the small volume of mucosa sucked into the suction cup such that the probe is fixed in place relative to the esophageal wall;
a fiber optic cable configured for delivering a laser beam to and through the probe and in the direction of the esophageal wall such that there is little or no relative movement between the laser beam and the esophageal wall;
wherein the probe is configured to measure laser Doppler perfusion indicative of the blood perfusion of the esophageal wall; and
a recorder for providing the laser beam and for receiving the measured laser Doppler perfusion measurements from the probe.

9. The system of claim 8 further comprising a pH sensor for measuring pH, wherein the recorder is further configured for receiving the pH measurements from the pH sensor.

10. The system of claim 8 further comprising an O₂ sensor for measuring O₂, wherein the recorder is further configured for receiving the O₂ measurements.

11. The system of claim 8 further comprising a CO₂ sensor for measuring CO₂, wherein the recorder is further configured for receiving the CO₂ measurement.

12. The system of claim 8 further comprising impedance rings for measuring impedance, wherein the recorder is further configured for receiving the impedance measurements.

13. The system of claim 8 further comprising an ultrasound transducer for making ultrasound measurements, wherein the recorder is further configured for receiving the ultrasound measurements.

14. The system of claim 8 further comprising an event marker button on the recorder for marking an event in time.

15. The system of claim 8 further comprising a computer interface for interfacing the recorder to a computer.
